(19) United States
(12) Patent Application Publication
(10) Pub. No.: US 2023/0372421 A1
(43) Pub. Date: Nov. 23, 2023

(54) IMMATURE POMEGRANATE EXTRACT FORMULATIONS

(71) Applicants: CONSIGLIO NAZIONALE DELLE RICERCHE, Romq (IT); VERA SALUS RICERCA S.r.l., Augusta (SR) (IT)

(72) Inventors: Laura SIRACUSA, Catania (IT); Carmelo DRAGO, Catania (IT); Giuseppe RUBERTO, Catania (IT); Giovanni Mario PITARI, Augusta (SR) (IT)

(73) Assignees: CONSIGLIO NAZIONALE DELLE RICERCHE, ROMA (IT); VERA SALUS RICERCA S.r.l., AUGUSTA (SR) (IT)

(21) Appl. No.: 18/030,455

(22) PCT Filed: Oct. 12, 2021
(86) PCT No.: PCT/IB2021/059338

§ 371 Date: Apr. 5, 2023

(30) Foreign Application Priority Data
Oct. 12, 2020 (IT) 102020000023920

(51) Int. Cl. A61K 36/185; A61K 31/365; A61K 31/366; A61P 35/00; A61K 45/06

(52) U.S. Cl. CPC A61K 36/185; A61K 31/365; A61K 31/366; A61P 35/00; A61K 45/06; A61K 2236/11; A61K 2236/15; A61K 2236/53; A61K 2236/333

(57) ABSTRACT

The present invention concerns an extract from pomegranate (Punica granatum L), specifically obtained from one or more immature pomegranate fruits. This extract is obtained through an extraction process including steps a)-d) as described in the text and it may contain gallotannins, ellagitannins, ellagic acid derivatives and granatins. Pharmaceutical and nutraceutical compositions and food supplements comprising this extract fall within the scope of the present invention as well as their use in various fields, more specifically for the treatment of tumors.

### AREA OF THE INVENTION

The present invention belongs to the area of extracts from vegetable matrices and their use in pharmaceutical and nutraceutical formulations.

Particularly, the present invention refers to pomegranate extracts.

### BACKGROUND AND STATE OF THE ART

Pomegranate (Punica granatum L.) is one of the most studied fruit matrices of the last decade has enjoyed a real boom as a ‘superfood’ product.

These interest and popularity can be explained by the particular characteristics of pomegranate juice and its related waste products; peels (Pathak, Mandavgane, & Kulkarni, 2017; Singh, Singh, Kaur & Singh, 2019; Andrade and others, 2019), seeds, mesocarp, i.e. the white spongy part that “holds” the arils where the juice is located, and other waste matrices obtained from pomegranate juice production have been extensively investigated in detail from a compositional point of view. (Bialek et al., 2019; Tortora and others, 2019).

Pomegranate products and by-products are also recognised to have a plethora of healing and health properties such as cancer prevention (Bassiri-Jahromi, 2018), treatment of metabolic disorders (Hou et al., 2019) and protection of the cardiovascular system (Wang and coworkers, 2018).

Little attention has so far been paid to pre-harvest pomegranate byproducts originating from the fruit thinning process. Fruit thinning, defined as the removal of excess flowers/clusters of flowers or individual fruitlets, is a common practice in horticulture, utilized to improve fruit size and quality. In pomegranate cultivation, the period of full bloom lasts about 1 month, and fruit set occurs in 2 or 4 distinct periods, with high quality fruits obtained in the early bloom (Shulman, Fainberestein, & Lavee, 1984). When excessive fruit set occurs, fruit thinning is recognized to improve pomegranate fruit size and quality (Jafari, Arzani, Fallahi & Barzegar, 2014; Kahramanoglu, Usanmaz & Alas, 2018).

Immature fruits from the fruit thinning process are therefore a waste product, requiring an often laborious and expensive waste management for local producers. In order to reduce the costs of waste disposal and apply a virtuous scheme of circular economy, the valorization and exploitation of these byproducts would be desirable.

Given the importance of pomegranate cultivation, the whole maturation process of pomegranate fruit to obtain a product (juice) commercially palatable and rich in bioactive constituents has been the object of many studies (Fawole & Opara, 2013 a-c). Nevertheless, only few of them have focused on the very early stages of fruit maturation.

As an example, Shwartz et al. (2009) reported the composition (as punicalagin, punicalin and ellagic acid content) of juices and peels from two different pomegranate cultivars monitored for 2 months, from a month before to a month after the harvest period, whilst Kulkarni & Aradhya (2005) studied the trend of total polyphenol contents in pomegranate arils during a wider time range. Similarly, Fawole & Opara (2013 a,b) reported on the physiological, chemical and qualitative changes in pomegranate juice cultivars “Bhagwa” and “Ruby” (South Africa) for 5 different maturation stages.

All the above publications deal with pomegranate fruit, arils and/or juice.

More recently, Nuncio-Jáuregui and coworkers (2015) described the pomological and chemical features, including simple sugars, punicalagin, ellagic acid and total polyphenol content of pomegranate immature fruits (35-40 days after the full tree bloom, when separation of arils from the rest of the fruit is not possible, as stated by the authors) of nine different cultivars of P. granatum. The authors regarded at these small fruits as a waste product, and mentioned the fruit thinning process. Moreover, they performed their analyses after fruit freeze-drying by extracting the lyophilized matrices with a hydro-alcoholic mixture. Freeze-drying is a time and cost consuming procedure, not appreciated in industry. The authors evaluated the total polyphenol content with the Folin-Ciocalteau method (results are reported as GAE, gallic acid equivalents), a colorimetric method giving approximate and often not reproducible values. Also, they used high performance liquid chromatography (HPLC) to assess the content of punicalagins (a and b) and ellagic acid, which are generally considered the principal pomegranate metabolites. No other molecules were reported in their study.

Therefore, there is the need to recycle immature pomegranate fruits using a time and cost-saving method, suitable for industrial scale-up. To do so, it is important to further explore the peculiar features and possible profitable uses of these immature fruits.

Here, it has been found that extracts obtained from immature pomegranate fruits through a specific extractive procedure contain a high amount of bioactive compounds and exhibit a higher antitumor activity than its ripe counterparts.

These findings open the way to a possible valorization of this pre-harvest byproducts of pomegranate production.

### OBJECT OF THE INVENTION

Thus, an object of the invention is an extract from pomegranate Punica granatum L. characterised in that it is obtained from one or more immature pomegranate fruits harvested from 30 to 40 days after fruit appearance or parts thereof by the following process:
(a) adding a sample of vegetable material comprising said one or more immature pomegranate fruits harvested 30 to 40 days after the appearance of the fruit or parts thereof an extraction medium in an amount sufficient to cover the vegetable material;
(b) homogenising the heterogeneous mixture of the vegetable material together with the extraction medium for a time period comprised between 3 minutes and 8 minutes;
(c) maintaining the suspension under mechanical stirring for 12 to 36 hours and/or exposing to ultrasounds for 15 to 90 minutes;
(d) separating the supernatant liquid from the solid residue in suspension to obtain the extract,
said extract comprising at least one compound belonging to the family of granatins.

Said extract is preferably obtained from peels of immature pomegranate.

In one embodiment, step c) is carried out in the dark and at room temperature.

In a preferred embodiment, said extraction medium used in step a) is selected from at least one alcohol, a combination of at least one alcohol and water, a combination of at least one alcohol, water and at least one acid and a biological serum, such as a dairy whey.

In one embodiment, the extract contains granatin B and/or its isomers.

In a preferred embodiment, the extract is characterized by the presence of gallotannins, ellagitannins and ellagic acid derivatives, particularly pedunculagin, punicalagins and granatins, particularly granatin B.

In one embodiment, the extract is characterized by a total polyphenol amount ranging from 60 to 400 mg/g of fresh vegetable material.

In particular, the extract is characterized by the presence of the following compounds in the indicated amounts:
punicalagins, a and b, in an amount comprised between 11 and 250 mg/g of fresh vegetable material;
total gallotannins in an amount comprised between 12 and 150 mg/g of fresh vegetable material, of which granatins in an amount comprised between 1 and 70 mg/g of fresh vegetable material;
ellagic acid derivatives in an amount comprised between 0.5 and 11 mg/g of fresh vegetable material.

Preferably, the amount of gallotannins with respect to total amount of polyphenols is comprised between 30 and 85% by weight, more preferably between 30 and 50% by weight.

Nutraceutical formulations or supplements containing this extract, alone or in combination with other extracts and components, preferably vegetable, fall within the scope of the present invention.

Pharmaceutical formulations containing such an extract, particularly as coadjuvant of drugs, are also covered by the present invention.

Dietary supplements containing this extract are also the subject of the present invention.

It is the object of the present invention the use of the extract or its pharmaceutical or nutraceutical composition or formulation or its dietary supplement in the treatment of tumors.

It is also the object of the present invention the use of the extract as a phytosanitary, disinfectant, pest control agent, antiseptic.

It is also the object of the present invention the use of said extract in the zoo-technical sector as a food or dietary supplement.

Moreover, it is the object of the present invention the use of said extract for the prevention, stabilization of clinical picture and/or the treatment of chronic diseases, developmental or aging pathologies, and neoplastic, cardiovascular, renal, hepatic, neurodegenerative, metabolic, endocrine diseases, hydro-electrolytic or bacterial flora imbalances, viral, bacterial or fungal infections, and inflammatory states.

Said extract can also be used as an antioxidant, antimicrobial, immunomodulator, anti-aging, regulator of hydroelectrolytic and/or tissue homeostasis, or as a food or dietary supplement, alone or in combination with a probiotic, a prebiotic or a symbiotic agent.

### DESCRIPTION OF THE INVENTION

### Figure

FIG. 1. HPLC chromatograms, displayed at 280 nm (nanometers), of the following pomegranate extracts A-F:
A: extract from mesocarp and arils of immature “baby red” pomegranate (BR-PA, 35 days from fruit set);
B: extract from immature “baby red” pomegranate peels (BR-B);
C: extract from mesocarp and arils of immature “baby green” pomegranate (BG-PA, 60 days from fruit set);
D: extract from immature “baby green” pomegranate peels (BG-B);
E: extract from mesocarp and arils of ripe pomegranate (M-PA);
F: extract from ripe pomegranate peels (M-B).

FIG. 2. Polyphenol content of the different pomegranate extracts. Polyphenol content quantified by HPLC is expressed in mg of metabolites/g of fresh vegetable material. Polyphenols are divided into chemical families (gallotannins, ellagitannins, etc.). Legend:
BR-PA: extract from mesocarp and arils of immature “baby red” pomegranate (35 days from fruit set)
BR-B: extract from immature “baby red” pomegranate peels;
BG-PA: extract from mesocarp and arils of immature “baby green” pomegranate (60 days from fruit set);
BG-B: extract from immature “baby green” pomegranate peels;
M-PA: extract from mesocarp and aryls of ripe pomegranate;
M-B: extract from ripe pomegranate peels.

FIG. 3. Antitumor effects of pomegranate extracts, the object of the invention. A) Proliferative heatmap of human lung (H1299) or colon (HCT116) adenocarcinoma cells treated for 48 hours (h) with 100 μg/mL of the indicated immature or ripe pomegranate extracts. Results (left panel) and grayscale (right panel) reflect percentages (%) to the reference controls treated with the vehicle DMSO. A1) Trend analysis of the anticancer effects of pomegranate peel extracts shown in A. **, p≤0.01. A2) Trend analysis of the anticancer effects of pomegranate mesocarp/aryl extracts shown in A.

FIG. 4. Inhibition of cancer cell proliferation by pomegranate extracts used in the examples. The bar graph illustrates antiproliferative effects (expressed as mean+/−SEM of percentage values compared to reference controls) in experiments (done in quadruplicate and repeated 3 times) conducted on human lung (H1299) or colon (HCT116) adenocarcinoma cells, treated for 48 hours (h) with 100 μg/mL of the indicated extracts of immature or mature pomegranate. CTR, vehicle-control. *, p≤0.05; **, p≤0.01 and ***, p≤0.001 vs vehicle-control reference.

### DETAILED DESCRIPTION OF THE INVENTION

It has now been found that extracts of immature pomegranate fruits, obtained with the extraction process described herein, are able to significantly reduce the hyperproliferative kinetics characteristic of cancer cells. This has been demonstrated, in particular, in two in vitro epithelial models, namely human lung and intestinal (colon) adenocarcinoma cells.

These results show biological potential of unripe fruit extracts of P. granatum as beneficial regulatory agents of altered proliferative kinetics in human cells.

This makes it possible the use of immature fruit extracts for therapeutic applications, which represents an advantageous opportunity for the commercial exploitation of these waste products from the pruning process.

Furthermore, the applied extraction process is simple, fast and economically convenient and therefore easily implementable in industrial settings.

The present invention refers to the extracts obtained from immature pomegranate fruits (Punica granatum L.). Any variant of pomegranate, in particular any species or cultivar, can be used to obtain the extract of the invention. In a particular embodiment these fruits belong to the “Wonderful” variety, but other varieties are equally suitable.

In the context of the present invention, “immature pomegranate fruit” means a fruit collected from the tree at about 30 to 60 days after fruit set. These days are also referred to as DAFB units (“days after fruit bloom”).

In one embodiment, the fruits may be in the state of ripeness of 35 days after fruit set, and may also be referred to as “baby red” or “baby red fruits”. In another embodiment, the fruits can be in the state of ripeness of 60 days from fruit set, and may also be referred to as “baby green” or “baby green fruits”.

In a preferred embodiment, the fruits are in the state of ripeness comprised between 30 and 40 days after fruit set, preferably 35 days after fruit set.

The extract can be obtained from any part of the fruit, in particular, the peel, arils and mesocarp. Preferably, the extract is obtained from the peel.

In one embodiment, the extract is obtained from any combination of parts of the fruit such as peel, arils and mesocarp, i.e. from peel and aryls or from peel and mesocarp or from aryls and mesocarp or from peel, arils and mesocarp.

In a particularly preferred embodiment, said extract is obtained from baby red fruit peels.

The extract according to the present invention is obtained through steps a)-d) of the method described above, which will be described in more detail as following.

In a first step a) an extraction medium is added to a sample of vegetable material in sufficient quantity to completely cover the vegetable material itself.
with the expression “sample of vegetable material” it is meant a fruit or more fruits, or one or more parts of them, of immature pomegranate, as defined above.

The said vegetable material is preferably fresh, e.g. dated between one and 10 days after actual harvesting.

After harvesting, the vegetable material is generally stored in a dry place preferably protected from direct light and heat.

In a particular embodiment, before beginning the extraction process, the vegetable material is chopped into small pieces, thus increasing the overall surface of the vegetable material exposed to the extraction medium.

The ratio between vegetable material and extraction medium is generally comprised between 1:2 and 1:8; preferably is 1:4.

Solvent is used here as a synonym for extraction medium.

The extraction medium can be any solvent suitable for extracting components from vegetable matrices.

Preferably, the extraction medium is an alcohol, a mixture of an alcohol and water, a mixture of an alcohol, water and an acid, or a biological serum.

As examples, the said alcohol can be methanol, ethanol, isopropanol or their mixtures.

Other alcohols are equally suitable.

In a preferred embodiment, a mixture of an alcohol and water is used, more preferably methanol and water or ethanol and water.

In the mixture, alcohol and water can be in any ratio. For example, the alcohol:water ratio could be comprised between 70:30 and 90:10, for example, it can be 80:20.

The water is preferably distilled water.

In an even more preferred embodiment, the said mixture of alcohol and water is added with a suitable amount of an acid, organic or inorganic, preferably selected from formic acid (HCO₂H), acetic acid (CH₃CO₂H), hydrochloric acid (HCl), citric acid (C₆H₈O₇), preferably formic acid. Other acids are equally suitable. In this embodiment the ratio of alcohol, water and acid can vary from 70:25:5 to 90:9.5:0.5, preferably the ratio is 80:19:1.

In a particularly preferred embodiment, the extraction is performed with a mixture of methanol/water/formic acid (MeOH/H₂O/HCO₂H) in a ratio of 80:19:1.

As biological serum it is meant a serum obtained from natural material. As an example, the said natural material could be milk. Biological serums are well known in the art and commonly used.

In a particular embodiment of the present invention, the extraction is carried out using biological serums suitable for food use, preferably but not exclusively comprising whey of various origins.

In step b) the mixture of the vegetable material and the solvent is subjected to homogenization.

The homogenization is an operation known in the industry through which a heterogeneous mixture is made as homogeneous as possible.

By heterogeneous mixture it is meant a mixture consisting of several phases, for example a solid and a liquid.

Homogenization can be carried out according to any method known in the sector and using equipment commonly used for this purpose.

In an embodiment the homogenization is mechanical and carried out using a mechanical homogenizer. An example of a mechanical homogenizer is an immersion blender. The dimensions of said blender will be suitable for the quantity of mixture to be homogenized.

In another embodiment, homogenization is achieved through the use of ultrasounds, by treating the mixture with equipment capable of generating ultrasounds. Such apparatuses are known in the art and commercially available.

Homogenization is advantageous as it allows the maximum surface of vegetable material to be exposed to the solvent or mixture of solvents.

Homogenization is carried out for a time between 3 to 8 minutes, for example for 3 minutes or for 5 minutes. After this time, a macroscopically homogeneous mixture of vegetable material and extraction solvent will be obtained.

In step c) the mixture obtained is left under mechanical stirring for an adequate time, for example for about 12 to 36 hours, for example for 18 hours.

Alternatively or in conjunction with mechanical stirring, the mixture obtained can be kept exposed to an ultrasound source for an adequate time, such as for about 15 minutes to 90 minutes, for example for 45 minutes. The use of ultrasounds accelerates the extraction process from the matrix, so that extraction with ultrasounds typically requires less time than that with mechanical stirring.

The extraction temperature can vary, although in general it can range from about 4 to about 30° C. Typically, it is carried out at room temperature, i.e. between 20 and 28° C.

Preferably, step c) is carried out in the dark and at room temperature.

In step c) a suspension formed by a solid residue of the vegetable material and a liquid supernatant is obtained.

Finally, in step d) the suspension is treated to separate the liquid supernatant from the solid residue. The liquid supernatant thus obtained represents the extract according to the invention.

For this purpose, any form of separation suitable and known in the art can be used, for example filtration or centrifugation.

In the case of separation by centrifugation, the mixture is centrifuged one or more times for a suitable time, e.g. from about 5 to about 10 minutes, at a suitable speed, e.g. at 3,000 to 10,000 rpm.

In some embodiments, the treatment of step d), for example centrifugation, is performed several times, each time recovering the liquid supernatant until no subsequent separation of a precipitate is observed. The collected fractions combined represent the extract of the invention.

The solid material in suspension can be re-extracted, then it can be subjected again to steps a)-d) described herein, using the same extraction medium or a different extraction medium to obtain further liquid extracts.

In a particular embodiment of the present invention, a weighted sample of fresh vegetable material is placed in a glass beaker and the extraction medium in sufficient quantity to cover the vegetable material is added. The heterogeneous mixture of the vegetable material and the extraction medium is then subjected to mechanical homogenization and subsequently, the mixture obtained is kept under stirring for an adequate time, typically for 12 to 36 hours, when the extraction is completed and the result is the re-separation of the phases, i.e. solid residue and liquid extract.

Optionally, the liquid extract can be further processed to obtain a solid, such as a paste or powder. In this embodiment, the extraction medium is removed from the liquid extract obtained at the end of step d) through, for example, methods of drying, distillation, dehydration or freeze-drying to obtain a pellet or a paste or a powder.

Said dried or freeze-dried extract is also an object of the present invention.

Preferably, the extraction is performed under sterile conditions.

In any one or more of the steps a)-d) the pH of the mixture can be adjusted by adding a basic compound or suitable buffer mixtures, as those well known in the art.

Said basic compound can be for example NH₄OH or NaOH.

Preferably, the pH is adjusted to a value comprised between about 5.5 and about 8.5.

In one embodiment, the extract of the present invention is characterized by the presence of an amount of polyphenols comprised between 60 and 400 mg/g of fresh vegetable material, preferably comprised between 250 and 400 mg/g of fresh vegetable material.

In one embodiment, the extract of the invention is characterized by the presence of at least one compound selected from the group of gallotannins, ellagitannins and ellagic acid derivatives. In a preferred embodiment, said extract comprises at least one compound belonging to the class of gallotannins, at least one compound belonging to the class of ellagitannins and at least one compound derived from ellagic acid.

In a preferred embodiment, the said compound belonging to the class of ellagitannins is selected among pedunculagin and punicalagin, in particular punicalagin a and/or b.

In a preferred embodiment, the said compound belonging to the class of gallotannins is a compound belonging to the family of granatins, in particular granatin B.

In one embodiment, the extract includes the following compounds in the following quantities:
punicalagin a and b, comprised between 11 and 250 mg/g of fresh vegetable material, preferably between 100 and 250 mg/g;
gallotannins, comprised between 10 and 150 mg/g of fresh vegetable material, preferably between 100 and 150 mg/g;
ellagic acid derivatives, in the range between 0.5 and 11 mg/g of fresh vegetable material, preferably between 5 and 11 mg/g;
Granatins, in the range between 1 and 70 mg/g of fresh vegetable material, preferably between 30 and 60 mg/g. The said granatins are preferably granatin B and/or its isomers.

In a preferred embodiment, the extract comprises granatin B and/or its isomers in an amount of between 1 and 70 mg/g of fresh vegetable material, preferably between 30 and 60 mg/g.

In an embodiment, the extract is characterized by a percentage of gallotannins with respect to the total polyphenol amount between 30 and 85%, preferably between 30 and 50%.

In one embodiment, the extract comprises one or more of the following compounds:
lagerstannin C (galloyl-HHDP-glucose) where HHDP stands for hexahydroxydifenic acid,
HHDP hexoside,
galloyl hexoside and/or its isomers,
galloyl-HHDP hexoside,
gallic acid,
punicalagin and/or its isomers or derivatives,
punicalin (gallagil hexoside) and/or its isomers,
di(HHDP-galloylglucose) pentoside,
pedunculagin and/or its isomers,
di-galloyl hexoside,
punicalagin a and/or b and/or their isomers,
pedunculagin III,
ellagic acid deoxy-hexoside,
punigluconin and/or its isomers,
pedunculagin II,
ellagic acid hexoside,
granatin B and/or its isomers,
ellagic acid galloyl hexoside and/or its isomers,
ellagic acid pentoside,
ellagic acid.

Preferably, the extract includes all of the following compounds:
galloyl hexoside,
gallic acid,
punicalin (gallagil hexoside) and its isomers,
punicalagin a,
pedunculagin and its isomers,
punicalagin b,
punigluconin and its isomers,
pedunculagin II,
ellagic acid hexoside,
granatin B and its isomers,
ellagic acid.

For isomer it is meant a compound having the same brute formula, i.e. the same molecular mass and percentage of atom composition.

A nutraceutical composition or formulation or a food supplement comprising the extract of the invention is also an object of the present invention.

Said nutraceutical formulation or food supplement can be employed for uses in humans or animals.

For nutraceutical composition or formulation it is meant an administrable preparation with beneficial effects on human health. Functional food is a synonym.

As food supplement it is meant a food product intended to supplement the common diet and which constitutes a source of substances having a nutritional or physiological effect. The said nutraceutical formulations may contain the extract object of the invention alone or in combination with other extracts from vegetable matrices. The extract of the invention may have obtained from only one part of the fruit or from several parts or from the whole fruit or from several fruits or parts of them.

The said additional extracts from vegetable matrices can be of any vegetable matrix suitable for food purposes, such as ginger, garlic, licorice, echinacea, Ginkgo biloba, turmeric, chilli, aromatic plants of the Labiatae family including sage and rosemary. Said extracts are known in the art, commercially available or easily obtainable accordingly to known methods.

The said nutraceutical or dietary supplement containing the extract of the invention can also be added to edible matrices, such as flour, milk, cheese, yoghurt.

The said nutraceutical or food supplement can be administered accordingly to any method known in the field, preferably it is administered orally or topically, for example as a syrup or other solid oral dosage forms or in microdispersed form by air, such as by aerosol.

The formulation of nutraceutical compositions and food supplements falls within the general knowledge of the expert in the field and does not require further specifications.

Preferably, the said nutraceutical composition or food supplement is intended for use in the prevention, stabilization of clinical parameters and/or the treatment of chronic diseases, developmental or aging disorders, neoplastic, cardiovascular, renal, hepatic, neurodegenerative, metabolic diseases, endocrine, hydro-electrolytic or intestinal microbiome imbalances, viral, bacterial or fungal infections, and inflammatory states. These pathologies are further detailed below.

By stabilization of the clinical parameters, it is meant the resolution of the main risk factors for the patient's health and the achievement of vital parameters consistent with a satisfactory existence.

The said nutraceutical can also be used for maintaining the state of health of healthy subjects and/or for restoring physiological well-being in a sick subject.

The said nutraceutical or food supplement can also be used as an antioxidant, that is, to slow down or prevent the oxidation of cellular components in a subject.

The said nutraceutical or food supplement can also be used as an antimicrobial or antibacterial agent for the elimination of microorganisms (viruses, bacteria, fungi) or to halt their proliferation.

The said nutraceutical or dietary supplement can also be used as an immunomodulatory, that is, able to regulate the immune response of a biological organism.

The said nutraceutical or food supplement can also be used as an anti-aging, that is, able to slow down or treat the aging of cells, tissues, organs.

The said nutraceutical or food supplement can also be used as a regulator of hydroelectrolytic or tissue homeostasis, for example for the treatment of diseases in which said hydroelectrolytic or tissue homeostasis is affected or altered.

The said nutraceutical or food supplement can also be used in combination with a probiotic, prebiotic or symbiotic agent, where with the term probiotic it is meant one or more microorganisms capable of exercising beneficial functions for humans or animals; by prebiotic it is meant a compound able to positively influence the growth and activity of one or more beneficial bacteria present in the human or animal intestine, and by symbiotic it is meant the combination of a probiotic and a prebiotic. Known probiotics, prebiotics and symbiotics can be used in combination with the extract of the invention. The expert in the field is able to choose a probiotic, prebiotic or symbiotic suitable for combination with the extract of the invention, depending on the desired use and intended activity.

The extract object of the invention can also be used as a phytosanitary, that is, a product suitable for the protection and/or defense of plants.

The use of the extract in the zoo-technical field as a food or dietary supplement for animals, in the field of environmental hygiene as a disinfectant or pest control agent, and as an antiseptic to protect human and animal health also fall within the scope of the present invention.

A pharmaceutical composition or formulation comprising the extract of the invention is also an object of the present invention.

The said pharmaceutical composition may include the extract of the invention alone or in combination with other vegetable extracts, such as ginger, garlic, licorice, echinacea, Ginkgo biloba, turmeric, chilli, aromatic plants of the Labiatae family including sage and rosemary.

In a preferred embodiment, said pharmaceutical composition is used as an adjuvant to one or more drugs.

As adjuvant we mean a compound or composition that completes or increases the effect of a substance, typically an active ingredient, from which the main effect is expected.

In particular, a pharmaceutical composition containing the extract of the invention can be used in combination with one or more pharmaceutical compositions including any other active principle. The two or more compositions can be used simultaneously or sequentially. In one embodiment, a single pharmaceutical composition comprises both the extract of the invention and one or more active pharmaceutical ingredients.

Said active principle, of which the extract of the invention is an adjuvant, can be for example: a chemotherapy or antitumor drug, an anti-inflammatory agent, an antiviral, an antibiotic, an antifungal, a senotherapeutic, a hormone, a biological agent, for example a monoclonal antibody, an antihypertensive, a diuretic, a neurotrophic, an angiogenesis regulator, an ion or electrolyte chelator, an hepatoprotector, a coagulation regulator, a regulator of body fluids.

Preferably, the said active principle is a chemotherapeutic or antitumor drug. In fact, the extract of the invention is particularly effective in reducing the proliferative activity of neoplastic cells, therefore it can be advantageously used as an adjuvant to an anticancer therapy. The said chemotherapy or anticancer drug can be used for the treatment of any tumor. For example, said chemotherapeutic or antitumor drug can be a compound chosen from alkylating agents, antimetabolites, antimitotics, topoisomerase I and II inhibitors, cytotoxic antibiotics, hormonal agents, enzymatic systems, immumodulators, anti-angiogenic agents, biological agents, molecule-targeted therapeutics.

The extract can be used for the prevention of pathological states, developmental or aging diseases, neoplastic, cardiovascular, renal, hepatic, neurodegenerative, metabolic, endocrine, hydro-electrolytic imbalances or bacterial flora, viral, bacterial or fungal, inflammatory states.

The extract of the invention can also be used for the stabilization of the clinical picture of diseases of aging, developmental pathologies or cardiovascular, renal, metabolic, neuroendocrine diseases, hydro-electrolytic imbalances or bacterial flora, viral, bacterial or bacterial infections inflammatory states.

The extract of the invention can also be used for the treatment of pathological diseases, developmental or aging diseases, neoplastic, cardiovascular, renal, hepatic, neurodegenerative, metabolic, endocrine diseases, hydro-electrolytic or intestinal microbiome imbalances, viral, bacterial or fungal infections, and inflammatory states.

Some uses of the extract that fall within the scope of the present invention will be described in more detail below.

By chronic disease it is meant a disease characterized by a slow and progressive decline of normal physiological functions. Illustrative but not exhaustive examples are respiratory diseases, some tumors, heart disease, the consequences of stroke, musculoskeletal disorders, neurological diseases, endocrinopathies, genetic, immune and idiopathic diseases, pathologies affecting sensory organs and chronic inflammatory diseases.

Developmental disease refers to a neurodevelopmental disorder that impairs or limits specific abilities of the brain. Illustrative but not exhaustive examples are learning disorders, dyslexia, attention deficit, autism spectrum disorders, mental deficits.

By aging disease it is meant the progressive acquisition with the advancement of the chronological age of chronic disabilities, often incurable, affecting one or more organs or biological systems of an individual. These are generally non-communicable diseases, such as cardiovascular, chronic obstructive pulmonary diseases, musculoskeletal diseases, diabetes, neurodegenerative diseases and tumors.

The neoplastic disease or tumor can be any tumor, in particular breast cancer, prostate cancer, renal cancer, pancreatic cancer, liver cancer, skin melanoma, uveal melanoma, myeloma, lung cancer, bowel cancer, ovarian cancer, bladder cancer, thyroid carcinoma, oligodendroglioma, glioblastoma, neuroblastoma, astrocytoma, neuroglioma, lipoma, angioma, sarcoma, leukemia, neuroendocrine tumors, lymphatic tumors, osteoblastoma, osteosarcoma, basal cell carcinoma, squamous cell carcinoma, mesothelioma.

The said viral infection can be, as examples, a human immunodeficiency virus (HIV) infection, SARS-CoV-2 (responsible for coronavirus disease 2019 or COVID-19), a coronavirus syndrome, hepatitis A-C, a herpes syndrome virus, a papillomavirus syndrome, rabies, flu, yellow fever, rubella, measles, chicken pox, smallpox, polio, enterocolitis, meningitis, encephalitis, pneumonia, an Ebola syndrome. Said bacterial infection can be tuberculosis, gonorrhea, pneumonia, encephalitis, enterocolitis, meningitis, otitis, pharyngitis, tonsillitis, salmonellosis, ulcer, gastritis, syndromes of the reproductive organs, urethritis, food poisoning, abscesses, purulent wounds, toxic shock. Said fungal infection can be histoplasmosis, candidiasis, mucomircosis, aspergillosis, blastomycosis, coccidioidomycosis, paracoccidioidomycosis, sporotrichosis.

The said neurodegenerative disease can be Parkinson's, Alzheimer's, Huntington's, Creutzfeldt-Jakob's disease, amyotrophic lateral sclerosis, vascular senile dementia, frontotemporal dementia, progressive supranuclear palsy, cerebellar ataxia.

The said metabolic disease can be diabetes, obesity, amyloidosis, gout, dyslipidemia syndrome, Gaucher disease, glycogenosis, leukodystrophy, lipodystrophy, mucopolysaccharidosis, steatoma, xanthomatosis.

The inflammation can be a general or local inflammatory state, such as allergic, atopic, immune, idiopathic, post-traumatic, infectious, post-infectious, reactive or secondary in nature.

The said pharmaceutical composition can be administered with any known method in the pharmaceutical sector.

Preferably, it is administered orally or topically.

For topical administration, the pharmaceutical composition can be preferably formulated as skin lotion, cream, essence, toner, emulsion, ointment, gelatin or suspension.

For oral administration, the pharmaceutical composition can be preferably formulated as syrup or any solid oral dosage form.

The pharmaceutical composition can also be formulated as a suspension for aerosol-like dosages.

The said pharmaceutical or nutraceutical formulations are able to provide easy administration with greater compliance and convenience for the patient, given the vegetal and biological nature of the extract of the invention. Furthermore, their use as nutraceuticals or food supplements, in addition to or in support of the normal daily diet, makes them particularly suitable for the regular and generalized intake by a subject.

The pharmaceutical compositions according to the present invention may also contain, in addition to the extract of the invention and any other vegetable extracts, suitable pharmaceutical vehicles and/or excipients. The selection of suitable carriers or excipients is within the capabilities of an expert in pharmaceutical arts. In general, pharmaceutically acceptable inert transporters preferably but not exclusively may include starch, mannitol, calcium sulfate, magnesium stearate, dicalcium phosphate, silica derivatives and sugars, including sucrose, lactose and glucose.

The excipients can be binding agents and diluents. The binding agents preferably but not exclusively may include carboxymethylcellulose and other cellulose derivatives, gelatin, natural and synthetic gums. Diluents generally include suitable oil, saline or sugar solutions and can be preferably but not exclusively selected from water, dimethyl isosorbide, ethylene glycol, diethylene glycol, triethylene glycol, propylene glycol, benzylene glycol, polyethylene glycol, ethyl alcohol, cetyl alcohol, glyceryl stearate, isopropyl alcohol, diethylamine, glyceryl oleate, myristyl alcohol, gelatin, simple syrup, cyclodextrin, polyvinyl pyrrolidone (Povidone), ethanol, maltitol, xylitol, inositol, mannitol, inverted sugars, sorbitol and the like or combinations thereof. Said diluent could also be preferably but not exclusively selected from glycerin, propylene glycol, sorbitol, water, ethanol and their combination.

Other excipients include lubricants, disintegrating agents and adsorption agents, all well known in the art. Coloring or flavoring agents can also be added to the composition of the present invention.

The pharmaceutical compositions of the present invention can be in the form of tablets, capsules, powders, suppositories, suspensions and solutions. The methods for making these various pharmaceutical formulations are within the competencies of a person skilled in the art.

The compositions of the present invention, which do not comprise pharmaceutical carriers or excipients can be in the form, as examples, of liquids, powders or tablets. The most basic form of the composition of the present invention is the liquid or solid product of the extraction process.

The compositions of the present invention contain at least a biologically effective amount of the extract of the invention. The biologically effective amount is considered to be that amount of the extract, in percentage by weight of the composition, which must be present to produce the desired biological or therapeutic effect. As would be apparent to a person skilled in the art, the biologically effective amount may vary, also depending on the condition to be treated and the form to be administered. In general, the extract will be present in an amount of about 1 to about 100% by weight of the composition. More specifically, the extract will be present in an amount ranging from 10 to 90% by weight, and even more specifically, from 20 to 80% by weight, and still more particularly, from 30 to 70% by weight, and even more particularly, from 40 to 60% by weight, and still more particularly, about 50% by weight of the composition.

The present invention also refers to the methods for making the pharmaceutical compositions of the appropriate extract, alone or in combination with other selected vegetable extracts.

The extracted liquid product obtained through the process object of the invention, or the dried product achieved from it, could be further combined with one or more pharmaceutical vehicles or excipients suitable to form the pharmaceutical compositions of the present invention. Suitable pharmaceutical carriers and excipients, as well as methods and conditions for manufacturing pharmaceutical formulations, are obvious to a person skilled in the art and they will not be discussed further.

The present invention also relates to a method for preparing the nutraceutical or pharmaceutical composition, which comprises the extract of vegetable material from the various parts, or from the whole of immature pomegranate fruits.

In one embodiment, the said method for preparing the pharmaceutical composition includes steps a)-d) of the method described above for obtaining the extract, and also the following steps:
(e) pH regulation of the extract in a range from 6 to 8;
(f) optionally repeating steps a)-e) and mixing the liquid extracts obtained;
(g) removing the extraction medium;
(h) mixing the liquid extract obtained with a suitable vehicle and/or pharmaceutical excipient.

In step e) the pH can be adjusted, as known in the art, by adding suitable compounds or buffer solutions.

In step g) the extraction medium can be removed by means of methods commonly known in the field, including drying, distillation, dehydration or lyophilization.

A food containing the extract of the invention alone or in combination with other vegetable extracts is also an object of the present invention. The said food can be flour, milk, yoghurt, soft drinks, fruit juice, cheese and the like. In this embodiment, the extract is added during the normal food preparation process.

This food can be used as food for special medical purposes.

The compositions of the present invention can be preferably but not exclusively administered orally, intranasally, rectally or parenterally. More particularly, suitable forms of parenteral administration include, but are not limited to, intravenous, subcutaneous, endotracheal, intra-arterial, endopleural, intrathecal, intramuscular and intraperitoneal injection.

The present invention also includes the encapsulation of the extract obtained with organic carriers, micro- and nano-particles or phospholipid liposomes to increase the correct delivery and bioavailability of the product. All such methods are known in the art and do not require further details.

As known to a person skilled in the art, the appropriate dosage for administration to a patient will depend on the disease being treated, the form of administration, the state of the disease and the particular characteristics of the patient, such as age and sex. For example, the extract can be administered orally at a daily dosage level between 10 mg/kg and 100 mg/kg of the patient's body weight.

The extract of the invention can also be included in a cosmeceutical composition or formulation.

As cosmeceutical composition is meant as a composition comprising one or more bioactive substances having both aesthetic and medical benefits. Said cosmeceutical composition can be obtained and used accordingly to what is known in the field.

The invention will also be described by the following illustrative examples.

### EXAMPLES

### Materials and Methods

### Vegetable Material

The vegetable material of the present invention consists of the fruits of 7-year-old pomegranate shrubs of the “Wonderful” variety, grown in an orchard located near Marsala (province of Trapani, Sicily; coordinates 37°52′12″N; 12°29′36″E) belonging to a local consortium of pomegranate producers. The shrubs were planted and then cultivated following the typical cultivation practices of commercial orchards (in this case 3.5×6 m spacing). The thinning process, which the vegetable material object of this invention comes from, was carried out from 5 to 9 weeks from the appearance of the fruit, specifically as follows: ten trees having an uniform flowering were randomly selected. On 5 of them the small unripe axillary fruits were removed by hand from the clusters, obtaining the first type of immature fruits studied (immature “baby red”, 35 days from fruit set), while on other 5 plants the immature fruits were removed at a more advanced stage (immature “baby green”, 60 days from fruit set). At commercial maturity of the fruit (October 2018, in this case) 15 ripe pomegranates were harvested from selected trees and sent to the laboratory for analysis.

### Cells and Reagents

Human lung (H1299) and intestinal (HCT116) adenocarcinoma cells were purchased from the American Type Culture Collection (ATCC; Manassas, VA, USA). Appropriate cell line authentication was provided by the vendor. Dulbecco's modified Eagle's medium (DMEM), RPMI 1640 medium, fetal bovine serum (FBS), and a 0.25% trypsin solution were obtained from Euroclone S.p.A. (Pero, Milan, Italy). Cells were propagated at 37° C. (5% CO₂) in medium enriched with 10% FBS, 100 units/mL of penicillin and 100 μg/mL of streptomycin (Invitrogen, Carlsbad, California, USA).

All reagents and solvents used in this work are of adequate purity for normal laboratory procedures, except water and HPLC grade acetonitrile purchased from VWR (Milan, Italy). Gallic acid, ellagic acid and punicalagin were purchased from Sigma Aldrich (Milan, Italy) and used as high purity standards.

### Extraction of Matrices and Preparation of Samples

The immature fruits of Punica granatum from two different thinning periods (baby red BR and baby green BG) as well as the ripe fruits (M), all ‘Wonderful’ cultivars, were carefully washed and dried with absorbent paper. Once cleaned, the fruits were manually separated in peel (exocarp, B) on one side and mesocarps and aryls (PA) on the other.

Variable aliquots (100 to 200 g) of these matrices: peels B and mesocarp and aryls PA from BR and BG fruits; peels B and mesocarp and aryls PA from M fruits, for a total of six different matrices (see Table 1) were separately extracted. Each aliquot was placed in a beaker and suspended with a weakly acid mixture of water and methanol (MeOH:H₂O:HCO₂H 80:19:1 v/v, about 400-500 mL), then homogenized in situ with a manual electric blender for about 3 minutes. The resulting heterogeneous mixture was further left under stirring by means of a magnetic stirrer at room temperature, protected from light for about 12-18 hours, then vacuum filtered through a büchner funnel equipped with absorbent paper already impregnated with solvent.

With this procedure, six different highly concentrated mother solutions were obtained from the six different matrices mentioned above. Small aliquots (1-2 mL) of these solutions were suitably diluted with HPLC grade water to obtain concentrations of about 5-12 mg fresh material/mL solvent, which were immediately transferred into 2 mL amber vials for HPLC and analyzed in real time. The remaining parts of the stock solutions were lyophilized (Lyoquest-85, Telstar Italy, Legnano, Milan, Italy), carefully weighed, and stored in a dry place. With this procedure the following freeze-dried matrices for biological assays were obtained (each matrix carries the same code according to the vegetable material from which it comes).

TABLE 1
Pomegranate fruit matrices (ripe and immature) analyzed in this work,
code used, weight after freeze-drying and yield from fresh fruit

| | | | | yield after |
|---|---|---|---|---|
| | | weight after | | freeze-drying |
| Matrix | code | freeze-drying | | from fresh fruit |
| mesocarp and arils of | BR-PA | 4.86 | gr | 6.1% |
| immature baby red (35 days | | | | |
| from fruit set) | | | | |
| peels from immature baby | BR-B | 10.31 | gr | 12.8% |
| red | | | | |
| Mesocarp and arils from | BG-PA | 4.67 | gr | 5.8% |
| immature baby green (60 | | | | |
| days from fruit set) | | | | |
| peels from immature baby | BG-B | 10.10 | gr | 12.6% |
| green | | | | |
| mesocarp and arils from | M-PA | 25 | gr | 4.6% |
| ripe pomegranate | | | | |
| (reference) | | | | |
| peels from ripe | M-B | 23 | gr | 23% |
| pomegranate (reference) | | | | |

### HPLC/DAD and HPLC/ESI-MS Analyses

The reported chromatographic analyses in this study were carried out on an “Ultimate3000 UHPLC focussed” instrument equipped with a high-pressure binary pump, a photodiode array detector, a thermostated column compartment and an autosampler injector (all Thermo Fisher Scientific, Inc., Milan, Italy). The data obtained were processed using the Chromeleon Chromatography Information Management System v. 6.80. The chromatographic runs were performed with a reversed phase column (Gemini C18, 250×4.6 mm, 5 μm particle size, Phenomenex Italia srl, Bologna, Italy) equipped with a pre-column (Gemini C18 4×3.0 mm, 5 μm particle size, Phenomenex Italia srl, Bologna, Italy). Metabolites of the pomegranate matrices were eluted using the following gradient of B (2.5% formic acid in acetonitrile) in A (2.5% formic acid in water): 0 min: 0% B; 25 min: 35% B; 30 min: 0% B. The solvent flow used was 1 mL/min, the temperature was kept at 25° C. throughout all analyses, and the selected injection volume was 10 μL. Quantification of metabolites was carried out at 280 nm (nanometers) for gallic acid and derivatives (HHDP, HHDP and derivatives, pedunculagin and granatins) using gallic acid as a reference (R2=0.9999). Quantification of ellagitannins was done at 360 nm using commercial punicalagin (98%) as standard (R2=0.9998) while ellagic acid was quantified together with its derivatives, again at 360 nm using ellagic acid as a reference (R2=0.9997). To unambiguously identify the chromatographic signals and to confirm peak assignments, HPLC/ESI-MS analyses were performed on the same matrices. In this case, the chromatographic apparatus used was the same as described above, while the Thermo Scientific Exactive Plus Orbitra MS ion trap equipped with a ESI electrospray interface (HESI II) (Thermo Fisher Scientific, Inc., Milan, Italy) was used as detector. Mass spectra were recorded operating in negative mode in the range from 120 to 1500 m/z with a resolving power of 25000 (full-width-at-half-maximum, at m/z 200, RFWHM), under the following operating conditions: capillary temperature: 300° C.; flow of nebulizer gas (nitrogen): 60 units; auxiliary gas flow: 10 units; source voltage: 3 kV; capillary voltage: 82.5 V; tube lens voltage: 85 V. The Orbitrap MS system was calibrated in positive mode using in direct infusion solutions of standard mixtures of SDS (sodium dodecyl sulfate, Mr 265.17 Da), sodium taurocholate (Mr 514.42 Da) and Ultramark (Mr 1621 Da). Data acquisition and analysis were carried out with the Excalibur software, supplied within the Orbitrap detector. All analyses were carried out in triplicate.

### Cell Proliferation

Immortalized tumor cells from lung (H1299, 1×10³) or intestinal (HCT116, 8×10²) epithelium were seeded in 96-well plates and grown for 72 h in their optimal culture media. Then, the cells were treated with 25, 50, 100 and 200 μg/mL of M-PA, MB, BG-PA, BG-B, BR-PA and BR-B (from DMSO stock solutions) for 48 h. Controls were treated with an equivalent vehicle volume (DMSO). At the end of the treatments, cells were fixed (in 4% paraformaldehyde) and stained with crystal violet (1%). After extraction of crystal violet with 10% acetic acid (for 10 min at room temperature), cell proliferation was quantified by measuring the absorbance at 590 nm with a spectrophotometer (Synergy HT, BioTek).

### Statistical Analysis

The results are shown as mean±SEM of 3 independent experiments performed in quadruplicate. Multiple comparisons were conducted with the One-Way ANOVA and Dunnett's post hoc test. Values of P were considered significant when α≤0.05. All statistical analyzes were carried out with the use of GraphPad Prism 8.0 (GraphPad Software, Inc., San Diego, CA).

### Example 1

Profiles of Secondary Metabolites from Different Parts of Ripe and Immature Pomegranate Fruits

This study reports in detail the composition in secondary metabolites, intended as a qualitative and quantitative characterization of the different parts (skin and pulp+arils) of the immature pomegranate fruits at 35 and 60 days from the appearance of the fruit, which were obtained as waste products from the thinning of pomegranate shrubs.

A series of HPLC/UV-VIS-DAD and HPLC/ESI-MS analyses were carried out in order to characterize the different parts of immature pomegranate fruits object of this study; the corresponding chromatograms are shown in FIG. 1.

Traces 1E and 1F resulting from the analyses on mesocarp and aryls (E) and peels (F) of ripe pomegranate fruits are reported as reference.

The use of H PLC/DAD and HPLC/ESI-MS techniques, as well as the comparison of the retention times reported in the literature, led to the identification of 32 different chromatographic signals, all belonging to the biochemical class of polyphenols, and particularly to gallotannins, ellagitannins and simple derivatives of ellagic acid. All these metabolites are widely recognized as part of the metabolic pool of pomegranate (Kharcoufi et al., 2018; Singh et al., 2018; Russo et al., 2018; Brighenti et al., 2017).

The two main subclasses of polyphenols present in these extracts are gallotannins and ellagitannins, which were preliminary characterized through the corresponding UV-VIS spectra (gallotannins with a single absorption band between 260 and 290 nm; ellagitannins with two distinct absorption bands, of which the diagnostic one at 370-380 nm). In this way, it was immediately possible to distinguish peaks from gallotannins (19 peaks), ellagitannins (7 peaks) and additional six peaks showing UV-vis spectra similar to that of ellagic acid, classified as ellagic acid-derivatives.

To identify individual peaks within the same subclass, it was necessary to use the information provided by the mass analyses (HPLC/ESI-MS) and specifically the data coming from TICs (total ion current) and SIMs (single ion monitoring). The results of the identification process are shown in Table 2.

TABLE 2
List of peaks and diagnostic data of selected metabolites from the pomegranate
by-products of this study. See FIG. 1 and the text for details.

| Peak | Rt, | | UV-vis data, | | |
|---|---|---|---|---|---|
| no. | min^{a} | compound identification | nm^{b} | MW | ESI⁻ data, m/z^{c} |
| 1 | 2.8 | lagerstannin C (galloyl-HHDP-glucose) | 238, 260sh | 650 | 649 (M − H)⁻, 301* |
| 2 | 3.8 | HHDP-hexoside | 258, 280 sh | 482 | 481 (M − H)⁻, 345* |
| 3 | 5.2 | galloyl hexoside | 279.4 | 332 | 331 (M − H)⁻ |
| 4 | 5.5 | galloyl-HHDP- hexoside | 278 | 634 | 633 (M − H)⁻, 301* |
| 5 | 6.0 | gallic acid^{d} | 280 | 170 | 169 (M − H)⁻ |
| 6 | 6.3 | punicalagin derivative | 269, 370 | 1124 | 1123(M − H)⁻, 1101 |
| 7 | 6.6 | punicalin (gallagyl-hexoside) | 259, 380 | 782 | 781(M − H)⁻, 601* |
| 8 | 6.9 | punicalin isomer | 259, 381 | 782 | 781(M − H)⁻ |
| 9 | 7.4 | di(HHDP-galloylglucose)-pentoside | 243.4, 268.2 | 1416 | 1415(M − H)⁻, 783* |
| 10 | 7.7 | punicalagin isomer | 243.8, 370 | 1084 | 1083 (M − H)⁻ |
| 11 | 7.8 | pedunculagin isomer 1 | 243.3 | 784 | 783(M − H)⁻, 601* |
| 12 | 8.2 | galloyl hexoside isomer | 280 | 332 | 331, 169* |
| 13 | 8.7 | digalloyl-hexoside | 240, 272 | 484 | 483(M − H)⁻, 271* |
| 14 | 9.4 | punicalagin a^{d} | 357.9, 378.2 | 1084 | 1083 (M − H)⁻, 601* |
| 15 | 9.9 | punicalagin isomer | 257, 378 | 1084 | 1083 (M − H)⁻ |
| 16 | 10.5 | pedunculagin III | 243.7 | 934 | 933(M − H)⁻, 721* |
| 17 | 10.9 | pedunculagin isomer 2 | 244.6, 264.6 | 784 | 783(M − H)⁻, 301* |
| 18 | 11.3 | punicalagin b^{d} | 257.9, 379 | 1084 | 1083 (M − H)⁻*, 601 |
| 19 | 12.2 | punigluconin isomer | 244.9 | 802 | 801(M − H)⁻, 347* |
| 20 | 12.9 | ellagic acid deoxy-hexoside | 254, 362 | 448 | 447(M − H)⁻, 301* |
| 21 | 13.6 | Punigluconin | 272.8 | 802 | 801(M − H)⁻, 649* |
| 22 | 14.3 | pedunculagin isomer 3 | 270 | 784 | 783(M − H)⁻, 301* |
| 23 | 14.8 | pedunculagin II | 269.7 | 786 | 785 (M − H)⁻, 633* |
| 24 | 15.0 | pedunculagin isomer 4 | 271.6 | 784 | 783(M − H)⁻, 765* |
| 25 | 15.3 | pedunculagin isomer 5 | 271 | 784 | 783(M − H)⁻, 765* |
| 26 | 15.8 | ellagic acid hexoside | 252, 360 | 464 | 463 (M − H)⁻, 301* |
| 27 | 16.4 | granatin B | 278 | 952 | 951(M − H)⁻, 933* |
| 28 | 16.9 | granatin B isomer | 278 | 952 | 951(M − H)⁻ |
| 29 | 17.8 | ellagic acid galloyl-hexoside | 253, 360 | 616 | 615(M − H)⁻, 301* |
| 30 | 18.6 | ellagic acid pentoside | 253, 358 | 434 | 433 (M − H)⁻* |
| 31 | 18.8 | ellagic acid galloyl-hexoside isomer | 253, 360 | 616 | 615(M − H)⁻* |
| 32 | 19.2 | ellagic acid^{d} | 252, 365 | 302 | 301(M − H)⁻* |

^{a}mean of six matrices × three replicates = 18 analyses;
^{b}from HPLC;
^{c}base peaks marked with an asterisk;
^{d}co-injection with pure commercial standard.

Qualitatively, all chromatograms shown in FIG. 1 are quite similar, with peaks 14 and 18, corresponding to punicalagin a and b anomers as main compounds in all fruit matrices. The only exception is given by the chromatographic analysis of the BR-PA matrix (FIG. 1A, mesocarp and arils from immature baby reds) where the peak number 17 (pedunculagin) is nearly as abundant as punicalagins a, and where peaks 21-26 (punigluconin and pedunculagin isomers, Table 2) are bigger than those in other chromatograms. Similarly, in the chromatogram corresponding to the BR-B matrix (immature baby red peels) peak 27 is unusually abundant and dominates the second part of the chromatogram (from 12 to 25 minutes, FIG. 1B). Peak 27 has been identified as granatin B, a gallotannin bearing a peculiar enantiomeric dehydrohexahydroxydiphenoyl (Okuda, Hatano, Nitta & Fujii, 1980; Steinmetz, 2010). Peak corresponding to granatin B is relevant also in BG-B, the peels from the ‘baby green’ pomegranate fruit (FIG. 1D).

### Example 2

### Secondary Metabolic Content of Different Parts of Pomegranate Immature and Ripe Fruits

After identification of the compounds belonging to the metabolic pool of pomegranate fruits, their quantification was performed. Results are reported in Table 3 and FIG. 2, where the compounds are for convenience divided into subclasses.

TABLE 3
Content of single metabolites from pomegranate matrices, reported in mg/g of fresh vegetable material.
metabolite content, mg/g fresh vegetable material^{a}

| Peak | | matrix^{b} | | | | | |
|---|---|---|---|---|---|---|---|
| # | compound | BR-PA | BG-PA | BR-B | BG-B | M-B | M-PA |
| 1 | lagerstannin C (galloyl-HHDP-glucose) | 0.057 | 0.037 | n.d. | n.d. | n.d. | 0.007 |
| 2 | HHDP hexoside | n.d. | 0.062 | 0.570 | 0.885 | 0.151 | 0.023 |
| 3 | galloyl hexoside | 0.065 | 0.049 | 0.252 | 0.595 | 0.301 | 0.039 |
| 4 | galloyl-HHDP hexoside | 0.118 | 0.130 | n.d. | 0.139 | 0.653 | 0.072 |
| 5 | gallic acid | 0.185 | 0.052 | 0.829 | 0.537 | 0.021 | 0.001 |
| 6 | punicalagin derivative | 0.010 | 0.015 | n.d. | n.d. | 0.159 | 0.006 |
| 7 | punicalin (gallagyl hexoside) | 0.038 | 0.068 | 1.016 | 0.999 | 0.398 | 0.034 |
| 8 | punicalin isomer | 0.040 | 0.073 | 1.298 | 1.090 | 0.621 | 0.043 |
| 9 | di(HHDP-galloylglucose) pentoside | 0.804 | n.d. | 3.303 | n.d. | n.d. | n.d. |
| 10 | punicalagin isomer | n.d. | 0.219 | 5.949 | 1.200 | 2.095 | 0.091 |
| 11 | pedunculagin isomer 1 | 1.296 | n.d. | 12.144 | 21.344 | n.d. | n.d. |
| 12 | galloyl-hexoside isomer | 0.323 | 0.104 | n.d | n.d | n.d. | n.d. |
| 13 | digalloyl hexoside | 0.091 | n.d. | n.d | n.d. | n.d. | n.d. |
| 14 | punicalagin a | 4.187 | 6.563 | 42.104 | 77.957 | 16.167 | 1.740 |
| 15 | punicalagin isomer | n.d. | n.d. | 14.693 | n.d. | n.d | n.d. |
| 16 | pedunculagin III | n.d. | 0.148 | 5.033 | 10.656 | 2.035 | 0.245 |
| 17 | pedunculagin isomer 2 | 12.360 | 4.974 | 17.166 | 13.035 | 0.419 | 0.073 |
| 18 | punicalagin b | 7.398 | 14.736 | 79.927 | 142.100 | 27.494 | 2.694 |
| 19 | punigluconin isomer | 3.578 | 2.720 | 17.913 | 11.428 | 2.947 | n.d. |
| 20 | ellagic acid deoxy-hexoside | n.d | n.d. | 0.017 | n.d. | n.d. | 0.001 |
| 21 | punigluconin | 1.454 | 0.327 | 3.564 | 2.360 | 0.727 | 0.039 |
| 22 | pedunculagin isomer 3 | 7.565 | 1.355 | 5.695 | 0.643 | n.d. | n.d. |
| 23 | pedunculagin II | 6.342 | 0.946 | 5.143 | 6.139 | 0.354 | 0.019 |
| 24 | pedunculagin isomer 4 | 7.290 | 1.736 | 10.058 | 9.950 | n.d. | 0.032 |
| 25 | pedunculagin isomer 5 | 1.534 | 0.211 | 2.852 | 4.387 | 0.179 | n.d. |
| 26 | ellagic acid hexoside | 0.304 | 2.171 | 1.916 | 4.525 | 3.316 | 0.340 |
| 27 | granatin B | 7.722 | 0.870 | 54.250 | 32.359 | 4.076 | 0.063 |
| 28 | granatin B isomer | n.d. | 0.218 | 5.510 | 4.219 | 0.771 | 0.036 |
| 29 | ellagic acid galloyl hexoside | n.d | 0.668 | n.d. | n.d. | 0.112 | 0.018 |
| 30 | ellagic acid pentoside | n.d. | 0.013 | 0.123 | 0.313 | 1.221 | 0.052 |
| 31 | ellagic acid galloyl hexoside isomer | n.d. | 0.015 | n.d. | 0.316 | 1.476 | 0.062 |
| 32 | ellagic acid | 0.491 | 0.398 | 5.012 | 5.759 | 1.803 | 0.089 |
| | total polyphenols | 63.25 | 38.88 | 296.34 | 352.94 | 67.50 | 5.82 |
| | punicalagin a + b | 11.59 | 21.30 | 122.03 | 220.06 | 43.66 | 4.43 |
| | total gallotannins | 43.06 | 12.85 | 84.52 | 82.10 | 7.79 | 0.55 |
| | total ellagic acid derivatives | 0.80 | 3.27 | 7.07 | 10.91 | 7.93 | 0.56 |
| | granatins | 7.72 | 1.09 | 59.76 | 36.58 | 4.85 | 0.10 |
| | total gallotannins (including granatins) | 50.78 | 13.94 | 144.28 | 118.68 | 12.63 | 0.65 |
| | % punicalagin over total polyphenols | 18.32 | 54.78 | 41.18 | 62.35 | 64.69 | 76.21 |
| | % gallotannins over total polyphenols | 80.29 | 35.85 | 48.69 | 33.63 | 18.72 | 11.15 |

BR = “baby red”, immature pomegranate fruit; BG = “baby green”, immature pomegranate fruit; M = ripe pomegranate fruit; PA = mesocarp and arils; B = peels. See FIG. 1 and text for details.
^{a}average of 3 replicates;
^{b}see text for details.

BG-B (peels from ‘baby green’ immature pomegranates) resulted the richest extract in polyphenols, with nearly 353 mg polyphenols/g of fresh fruit material, followed by BR-B (peels from ‘baby red’ immature pomegranates) with 296 mg/g of fresh material. The remaining extracts presented comparatively modest phenolic contents, with highest amounts in M-B (peels from mature pomegranates) containing 67.5 mg polyphenols/g of fresh material, and BR-PA (mesocarp and arils from ‘baby red’ immature pomegranates) with 63.2 mg/g of fresh material. For all samples, the amount of polyphenols was higher in peels than in mesocarp and arils, as already reported (Singh et al., 2018). The anomers punicalagin a and b were confirmed as the most abundant metabolites in all matrices except BR-PA, in which total gallotannin amounts were the highest (FIG. 2 and Table 3).

In addition, granatins (granatin B and its isomer, peaks 27 and 28, FIG. 1 and Table 2) were confirmed to be particularly abundant only in BR-B (nearly 60 mg/g of fresh fruit material) and BG-B (36.5 mg/g of fresh material), the peels from unripe fruits (Table 3).

As a general trend, a noticeable decrease in phenolic content was observed from immature fruits ‘baby red’ and ‘baby green’ to ripe pomegranates. This result is a very well documented phenomenon in pomegranate, which contributes to the astringency loss and sweet taste gaining of mature fruits (Shwartz et al. 2009; Kulkarni & Aradhya, 2005), together with the appearance of the typical color of the aryls given by the anthocyanins which are predicted to be present only in the two reference samples (MB and M-PA).

### Example 3

Effects on Cell Proliferation Kinetics of Pomegranate Extracts from Immature and Ripe Fruit

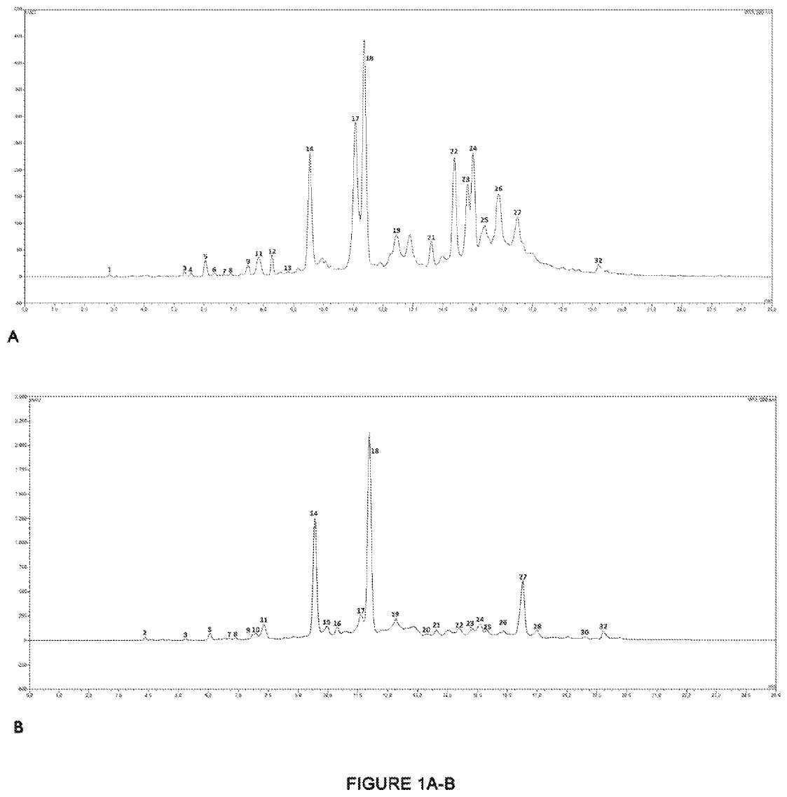

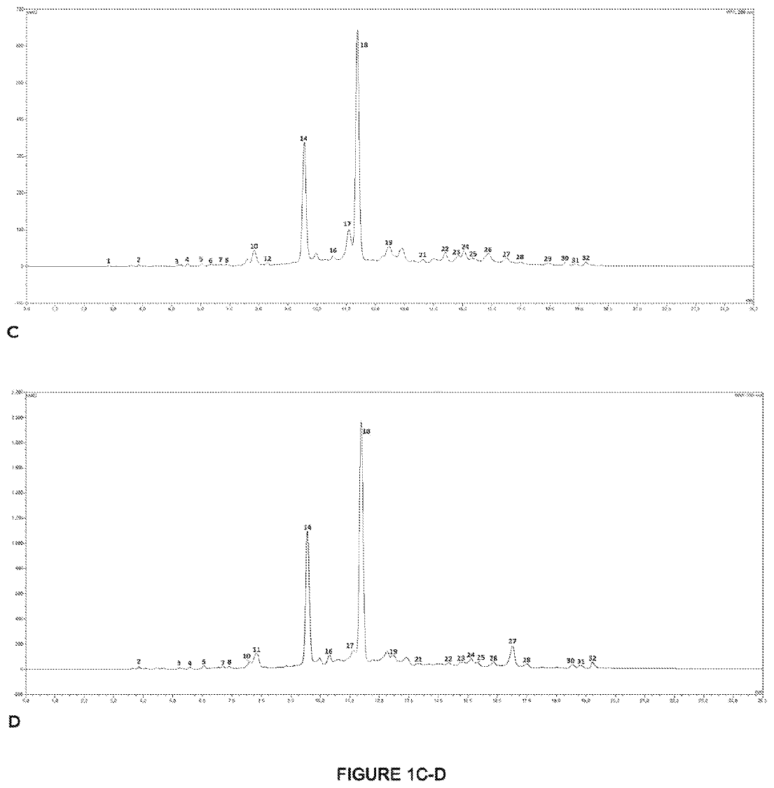

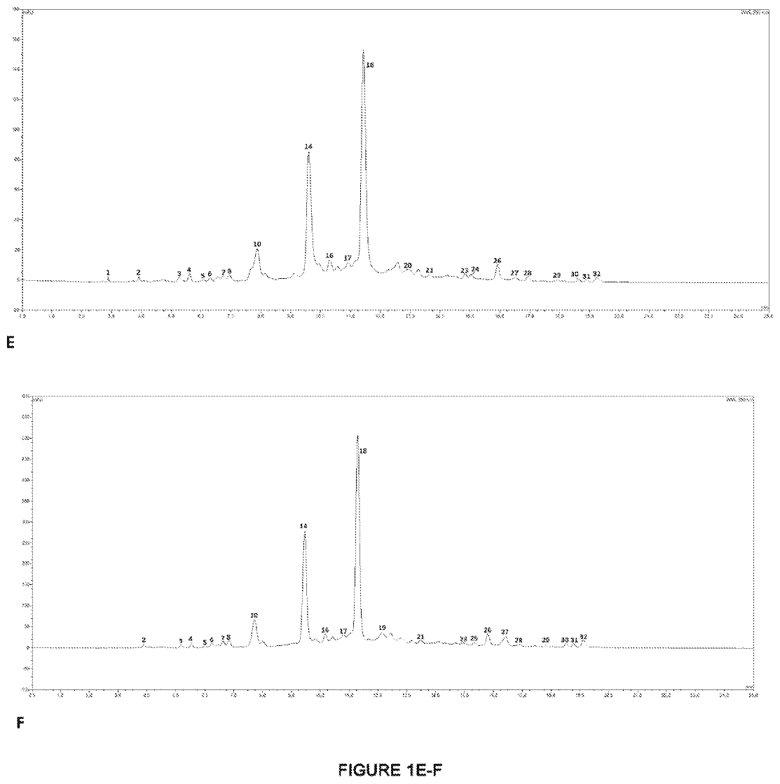

To define the biological properties of pomegranate extracts at different stages of maturation, their ability to regulate cell proliferation kinetics in two different epithelial cell models (human H1299 lung and HCT116 intestinal adenocarcinomas) was examined in vitro. Results are shown in Table 4 and FIG. 3.

TABLE 4
Statistical comparisons of antiproliferative activities by different
extracts from pomegranate fruits in H1299 and HCT116 cells.

| | H1299 | | | HCT116 | | |
|---|---|---|---|---|---|---|
| Pomegranate | 95.00% CI | | | 95.00% CI | | |
| Extracts | of diff | P Value | Significance | of diff | P Value | Significance |
| BR-B vs. BR-PA | 14.53 | 0.002 | ** | 44.51 | <0.001 | *** |
| | to 54.44 | | | to 84.42 | | |
| BR-B vs. BG-B | 36.17 | 0.11 | Ns | −48.69 | 0.007 | ** |
| | to 3.745 | | | to −18.772 | | |
| BR-B vs. BG-PA | −46.07 | 0.01 | * | −77.62 | <0.001 | *** |
| | to −6.154 | | | to −37.71 | | |
| BR-B vs. M-B | −51.34 | 0.003 | ** | −72.73 | <0.001 | *** |
| | to −11.43 | | | to −32.81 | | |
| BR-B vs. M-PA | −60.89 | <0.001 | *** | −75.48 | <0.001 | *** |
| | to −20.98 | | | to −35.57 | | |
| BR-PA vs. BG-PA | −11.58 | 0.39 | ns | −13.16 | 0.49 | ns |
| | to 28.33 | | | to 26.76 | | |
| BG-B vs. BG-PA | −10.06 | 0.32 | ns | 8.983 | 0.006 | ** |
| | to 29.86 | | | to 48.90 | | |
| BG-B vs. BR-PA | −1.680 | 0.07 | ns | 15.78 | 0.001 | ** |
| | to 38.23 | | | to 55.70 | | |
| BG-B vs. M-B | −35.13 | 0.13 | ns | −44.00 | 0.02 | |
| | to 4.784 | | | to −4.086 | | |
| BG-B vs. M-PA | −44.68 | 0.02 | * | −46.75 | 0.01 | * |
| | to −4.769 | | | to −6.838 | | |
| M-B vs. M-PA | −10.40 | 0.33 | ns | −17.20 | 0.78 | ns |
| | to 29.51 | | | to 22.71 | | |
| M-B vs. BR-PA | −16.85 | 0.75 | ns | −8.260 | 0.24 | ns |
| | to 23.06 | | | to 31.65 | | |
| M-B vs. BG-PA | −25.23 | 0.59 | ns | −15.06 | 0.62 | ns |
| | to 14.68 | | | to 24.85 | | |
| M-PA vs. BR-PA | −26.41 | 0.51 | ns | −11.01 | 0.36 | ns |
| | to 13.51 | | | to 28.90 | | |
| M-PA vs. BG-PA | −34.78 | 0.14 | ns | −17.81 | 0.83 | ns |
| | to 5.130 | | | to 22.10 | | |

BR = immature “baby red” pomegranate fruit; BG = immature “baby green” pomegranate fruit; M = ripe pomegranate fruit; PA = mesocarp and aryls; B = peels.
Ns, not significant.

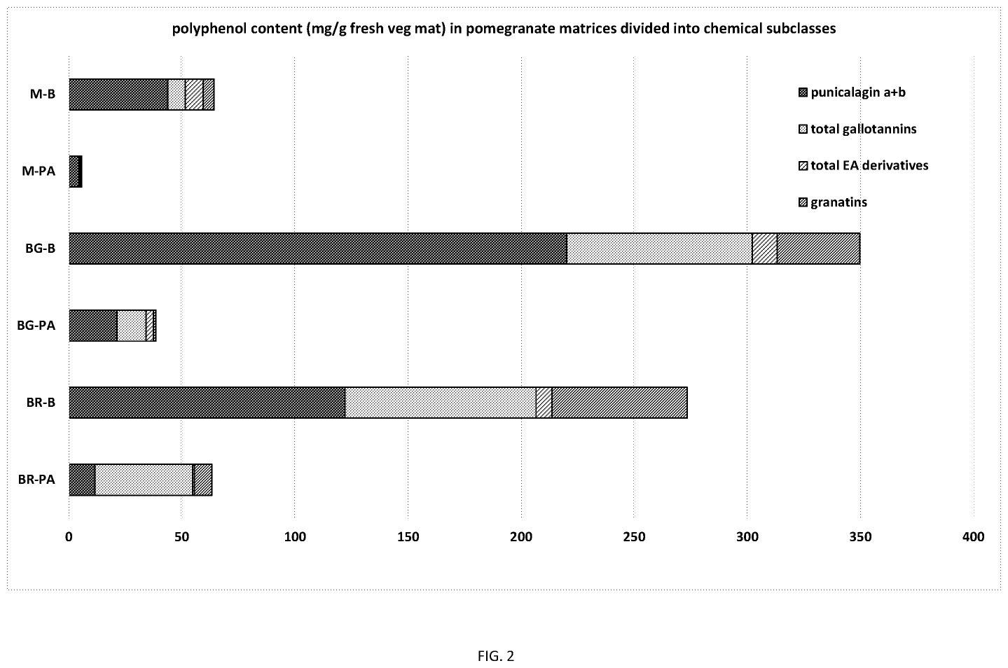

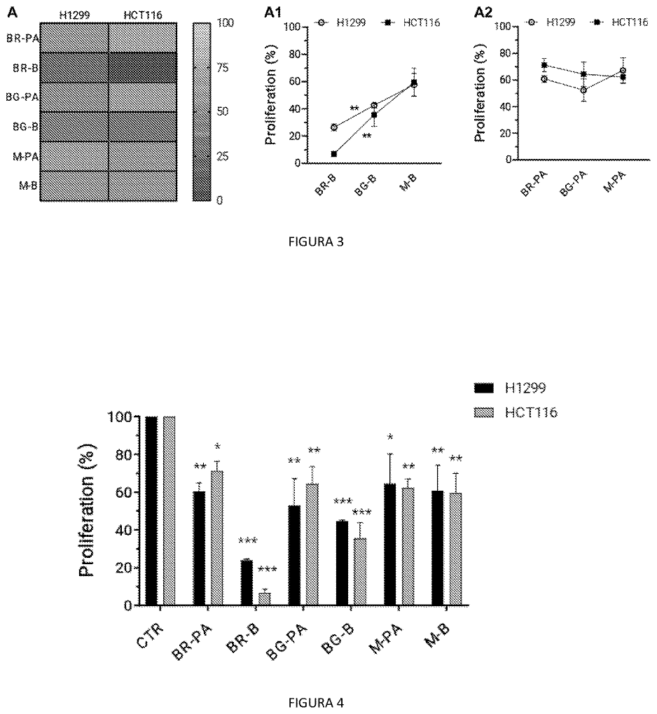

Compared to the respective controls (treated with the vehicle), all the extracts (beyond the maturation stage) significantly reduced the hyperproliferative kinetics characteristic of tumor cells (FIG. 4).

Notably, the differences in antiproliferative efficacy between pomegranate peels (B) and mesocarp and aryls (PA) exhibited a ripening gradient (FIG. 3A) with the largest, significant differential effects shown by the immature baby red (BR) fruit, and the minor, insignificant difference present in the mature (M) fruit.

Moreover, effects on proliferation were higher in peel extracts (B) from immature pomegranate fruits, compared to all the other parts (FIG. 4).

In general, peel extracts (B) showed the most pronounced effects compared to the respective parts derived from mesocarp and aryl (PA; FIG. 3), probably as a consequence of the different amounts of polyphenols contained in the two organic matrices.

These results reveal a biological potential of unripe fruit extracts of P. granatum as regulators of the altered proliferative kinetics in human epithelial cells, underscoring the high value and commercial significance of these waste products from pomegranate thinning processes.

Compared to all other extracts, BR-B induced the most pronounced and significant effects in both human cell lines examined, with the sole exception of BG-B in H1299 cells (Table 4). BG-B, in turn, showed significantly reduced proliferative rates compared to all other extracts only in HCT116 (Table 4), indicating the probable presence of cell type-specific susceptibility differences.

These experimental observations indicate that extracts of immature pomegranate fruits, and especially of baby red, are a rich natural source of metabolites with biological activities as regulators of proliferative kinetics in human cells.

Based on results from compositional analyses (Table 3), it is possible to speculate that granatins might substantially contribute to those antiproliferative effects. In fact, granatins are the most abundant in peels and show an inverse concentration gradient from BR-B to BG-B and M-B (Table 3), that mirrors the antiproliferative trend exhibited by pomegranate peel extracts (FIG. 3A1). Furthermore, granatin B, the most abundant granatin in peel extracts from unripe fruits (FIGS. 1-2 and Table 3), has been described to possess important anticancer properties in cells of neuronal origin (Jin, Yu, Jin, Wang & Xu, 2016). Therefore, granatin B represents a promising candidate for future translational developments in the nutraceutical and pharmaceutical sectors.

In conclusion, the compositional study (in terms of secondary metabolites) of immature pomegranate fruits at two different ripening stages, considered waste products from shrub thinning procedures, highlights a high content of biologically active polyphenols, mainly gallotannins and ellagitannins.

The results from assays on immortalized epithelial cells of tumor origin (adenocarcinomas of the lung and colon) demonstrate strong normalization activities toward altered proliferative kinetics by extracts from immature pomegranate fruits according to the present invention, which supports their utility in nutraceutical and pharmaceutical formulations or dietary supplements with specifically beneficial applications in the nutraceutical, cosmeceutical and food sectors.

### BIBLIOGRAPHY

Andrade, M. A., Lima, V., Sanches Silva, A., Vilarinho, F., Castilho, M. C., Khwaldia, K., Ramos, F. (2019). Pomegranate and grape by-products and their active compounds: Are they a valuable source for food applications? Trends in Food Science & Technology, 86, 68-84.
Bassiri-Jahromi, S. (2018). Punica granatum (pomegranate) activity in health promotion and cancer prevention. Oncology Reviews, 12, 345.
Bialek, M., Bialek, A., Lepionka, T., Paśko, P., Galanty, A., Tokarz, A., Czauderna, M. (2019). Punica granatum (Pomegranate) Seed Oil and Momordica charantia (Bitter Melon) Extract Affect the Lipid's Profile and Oxidative Stability of Femoral Muscles of Rats. European Journal of Lipid Science and Technology, 121, 1800420 (1-7).
Brighenti, V., Groothuis, S. F., Prencipe, F. P., Amir, R., Benvenuti, S., Pellatia, F. (2017). Metabolite fingerprinting of Punica granatum L. (pomegranate) polyphenols by means of high-performance liquid chromatography with diode array and electrospray ionization-mass spectrometry detection. Journal of Chromatography A, 1480, 20-31.
Fawole, O. A., Opara, U. L. (2013a). Effects of maturity status on biochemical content, polyphenol composition and antioxidant capacity of pomegranate fruit arils (cv. ‘Bhagwa’). South African Journal of Botany 2013,85,23-31.
Fawole, O. A., Opara, U. L. (2013b). Changes in physical properties, chemical and elemental composition and antioxidant capacity of pomegranate (cv. Ruby) fruit at five maturity stages. Scientia Horticulturae, 150, 37-46.
Fawole, O. A., Opara, U. L. (2013c). Developmental changes in maturity indices of pomegranate fruit: A descriptive review. Scientia Horticulturae, 159, 152-161.
Hou, C., Zhang, W., Li, J., Du, L., Lv, O., Zhao, S., Li, J. (2019). Beneficial Effects of Pomegranate on Lipid Metabolism in Metabolic Disorders. Molecular Nutrition and Food Research, 1800773.
Jafari, A., Arzani, K., Fallahi, E., Barzegar, M. (2014). Optimizing Fruit Yield, Size, and Quality Attributes in ‘Malase Torshe Saveh’ Pomegranate through hand Thinning. Journal of the American Pomological Society, 68(2), 89-96.6
Jin, Z., Yu, Y., Jin, R.-H., Wang, Y.-B., Xu, H.-Y. (2016). Effect of granatin B on the glioma cancer by inducing apoptosis. American Journal of Translational Research, 8(9), 3970-3975.
Kharcoufi, S., Licciardello, F., Siracusa, L., Muratore, G., Hamdii, M., Restuccia, C. (2018). Antimicrobial and antioxidant features of ‘Gabsi’ pomegranate peel extracts. Industrial Crops and Products, 111, 345-352.
Kulkarni, A. P., Aradhya, S. M. (2005). Chemical changes and antioxidant activity in pomegranate arils during fruit development. Food Chemistry, 93, 319-324.
Larrosa, M., Tomás-Barberán, F. A., & Espin, J. C. (2006). The dietary hydrolysable tannin punicalagin releases ellagic acid that induces apoptosis in human colon adenocarcinoma Caco-2 cells by using the mitochondrial pathway. The Journal of Nutritional Biochemistry, 17(9), 611-625.
Nuncio-Jäuregui, N., Munera-Picazo, S., Calin-Sánchez, A., Wojdylo, A., Hernãndez, F., Carbonell-Barrachina, Á. A. (2015). Bioactive compound composition of pomegranate fruits removed during thinning. Journal of Food Composition and Analysis, 37, 11-19.
Okuda, T., Hatano, T., Nitta, H., Fujii, R. (1980). Hydrolysable tannins having enantiomeric dehydrohexahydroxydiphenoyl group: Revised structure of terchebin and structure of granatin B”. Tetrahedron Letters, 21, 4361-4364.
Pathak, P. D., Mandavgane, S. A., & Kulkarni, B. D. (2017). Valorization of pomegranate peels: A biorefinery approach. Waste and Biomass Valorization, 8(4), 1127-1137.
Russo, M., Fanali, C., Tripodo, G., Dugo, P., Muleo, R., Dugo, L., De Gara, L., Mondello, L. (2018). Analysis of phenolic compounds in different parts of pomegranate (Punica granatum) fruit by HPLC-PDA-ESI/MS and evaluation of their antioxidant activity: application to different Italian varieties. Analytical and Bioanalytical Chemistry, 410, 3507-3520.
Shulman, Y., Fainberestein, L., Lavee, S. (1984) Pomegranate fruit development and maturation. Journal of Horticultural Science, 59, 265-274.
Shwartz, E., Glazer, I., Bar-Ya'akov, I., Matityahu, I., Bar-Ian, I., Holland, D., Amir, R. (2009). Changes in chemical constituents during the maturation and ripening of two commercially important pomegranate accessions. Food Chemistry, 115, 965-973.
Singh, B., Singh, J. P., Kaur, A., Singh, N. (2018). Phenolic compounds as beneficial phytochemicals in pomegranate (Punica granatum L.) peel: A review. Food Chemistry, 261, 75-86. DOI: https://doi.org/10.1016/j.foodchem.2018.04.039.
Steinmetz, W. E. NMR assignment and characterization of proton exchange of the ellagitannin granatin B (2010). Magnetic Resonance In Chemistry, 48, 565-570.
Tortora, K., Femia, A. P., Romagnoli, A., Sineo, I., Khatib, M., Mulinacci, N., Giovannelli, L., Caderni, G. (2018). Pomegranate By-Products in Colorectal Cancer Chemoprevention: Effects in Apc-Mutated Pirc Rats and Mechanistic Studies In Vitro and Ex Vivo. Molecular Nutrition and Food Research, 62, 1700401.
Wang, D., Özen, C., Abu-Reidah, I. M., Chigurupati, S., Patra, J. K., Horbanczuk, J. O., Jó\tilde{z}wik, A., Tzvetkov, N. T., Uhrin, P., Atanasov, A. G. (2018).Vasculoprotective Effects of Pomegranate (Punica granatum L.). Frontiers in Pharmacology, 9, 544.

1. An extract from pomegranate Punica granatum L. obtained from one or more immature pomegranate fruits harvested from 30 to 40 days after an appearance of the fruit or parts thereof by the following process:
a) adding to a sample of vegetable material comprising said one or more immature pomegranate fruits harvested from 30 to 40 days after the appearance of the fruit or parts thereof an extraction medium in an amount sufficient to cover the vegetable material;
b) homogenizing the heterogeneous mixture of the vegetable material with the extraction medium for a time period comprised between 3 and 8 minutes;
c) maintaining the suspension under mechanical stirring from 12 to 36 hours and/or ultrasounds exposure from 15 to 90 minutes;
d) separating the supernatant liquid from the solid residue in suspension to obtain the extract, said extract comprising at least one compound belonging to the family of granatins.

2. The extract according to claim 1, wherein said extract is obtained from peels of said immature pomegranate fruits.

3. The extract according to claim 1, wherein step c) is carried out in the dark and at room temperature.

4. The extract according to claim 1, wherein said extraction medium used in step a) is: an alcohol, a mixture of an alcohol and water, a mixture of an alcohol, water, an acid and a biological serum, or a mixture MeOH/H₂O/HCO₂H in a ratio 80:19:1.

5. The extract according to claim 1, further comprising at least granatin B and/or its isomers.

6. The extract according to claim 1, further comprising:
Punicalagin, α and β, in an amount comprised between 11 and 250 mg/g of fresh vegetable material;
Gallotannins in an amount comprised between 10 and 150 mg/g of fresh vegetable material;
Ellagic acid derivatives in an amount comprised between 0.5 and 11 mg/g of fresh vegetable material, and
Granatins in an amount comprised between 1 and 70 mg/g of fresh vegetable material.

7. The extract according to claim 6, wherein the amount of gallotannins with respect to a total amount of polyphenols is comprised between 30 and 85% by weight, preferably between 30 and 50% by weight.

8. An extract in the form of a pellet or of a paste or of a powder obtained by drying, distillation, dehydration or lyophilization of the extract of claim 1.

9. A pharmaceutical or nutraceutical composition or formulation or a dietary supplement containing the extract of claim 1, alone or in combination with other extracts and/or vegetable components.

10. Use of the pharmaceutical or nutraceutical composition or formulation or dietary supplement of claim 9 for the treatment of tumors.

11. Use of the pharmaceutical or nutraceutical composition or formulation or dietary supplement of claim 9:
a) as an coadjuvant of one or more drugs and/or active pharmaceutical ingredients; and/or
b) in combination with one or more pharmaceutical compositions comprising a chemotherapeutic agent.

12. The pharmaceutical or nutraceutical composition or formulation or dietary supplement of claim 9 for use for the prevention, the stabilization of the clinical picture and/or the treatment of chronic diseases, developmental or aging pathologies, neoplastic, cardiovascular, renal, hepatic, neurodegenerative, metabolic, endocrine diseases, hydro-electrolytic or bacterial flora imbalances, viral, bacterial or fungal infections, or inflammatory states.

13. Use of the pharmaceutical or nutraceutical composition or formulation or dietary supplement of claim 9 as antioxidant, antimicrobial, immunomodulator, anti-aging, regulator of hydro-electrolytic and/or tissue homeostasis, or in combination with a probiotic, a prebiotic and/or a symbiotic, alone or added to edible matrices.
